(19) United States
(12) Patent Application Publication
(10) Pub. No.: US 2015/0148417 A1
(43) Pub. Date: May 28, 2015

(54) SYNTHESIS AND BIOLOGICAL STUDIES OF AN ISOMERIC MIXTURE OF (E/Z) ISOXYLITONES AND ITS ANALOGUES

(71) Applicants: Atta-ur- Rahman, Karachi (PK); M. Iqbal Choudhary, Karachi (PK); Farzana Shaheen, Karachi (PK); Shabana Usman Simjee, Karachi (PK); Noureen Kahn, Karachi (PK); Saima Mahmood Malhi, Karachi (PK); Syed Uzair Ali Shah, Karachi (PK); Muhammad Nadeem Ashraf, Karachi (PK)

(72) Inventors: Atta-ur- Rahman, Karachi (PK); M. Iqbal Choudhary, Karachi (PK); Farzana Shaheen, Karachi (PK); Shabana Usman Simjee, Karachi (PK); Noureen Kahn, Karachi (PK); Saima Mahmood Malhi, Karachi (PK); Syed Uzair Ali Shah, Karachi (PK); Muhammad Nadeem Ashraf, Karachi (PK)

(21) Appl. No.: 14/609,211

(22) Filed: Jan. 29, 2015

Related U.S. Application Data
(63) Continuation of application No. 13/758,027, filed on Feb. 4, 2013.

(51) Int. Cl. A61K 31/19

(52) U.S. Cl. CPC A61K 31/19
USPC 514/557

(57) ABSTRACT

The invention relates to an anti-epileptic isoxylitone, 2,2′-(3,5,5-Trimethyl-2-cylohexen-1-ylidene)acetic acid.

### CROSS REFERENCE TO RELATED APPLICATION

This application is a continuation and claims the priority benefit of U.S. patent application Ser. No. 13/758,027, entitled “SYNTHESIS AND BIOLOGICAL STUDIES OF AN ISOMERIC MIXTURE OF (E/Z) ISOXYLITONES AND ITS ANALOGUES” filed on Feb. 4, 2013, and incorporated herein by reference in its entirety.

### BACKGROUND OF THE INVENTION

Epilepsy is a major neurological disorder globally, with high prevalence in developing world. About 30% of the epileptic population has seizures that are not responsive to presently available medical therapies. 90% of the people with epilepsy are found in developing regions. The currently available antiepileptic drugs are generally synthetic in nature. Despite many available chemotherapeutic agents, none are capable of controlling the seizures completely and most drugs have severe side-effects. In view of the large percentage of uncontrolled epileptics and the side effects experienced by patients with the existing medications, there is an urgent need for more selective and less toxic anticonvulsant drugs.

Recent studies in our laboratory have led to the discovery of potent anticonvulsant agents, isoxylitones A and B from a medicinal plant Delphinium denudatum Wall. Bioassay-guided isolation studies on this plant were carried out to isolate anticonvulsant constituents of this plant. The crude ethanolic extract of this plant was subjected to bioassay-guided fractionation which revealed that chloroform extracts containing diterpenoid alkaloids were highly toxic to neuromuscular system of mice. It was found that anticonvulsant constituents were found in least toxic of all extracts, the non-alkaloidal aqueous extract of plant. The aqueous extract was further subjected to vacuum liquid chromatography which afforded non-toxic and non-alkaloidal oily material which exhibited strong anticonvulsant activities in in vivo animal models of epilepsy, such as maximal electroshock test (MEST), and subcutaneous pentylenetetrazole (scPTZ). In in vitro studies, FS-1 strongly inhibited SRF of neurons at a dose of 0.06 mg/ml. The oily fraction strongly inhibited PTZ-induced epileptiform activity of hippocampal neurons in culture cells. Further purification of oily material led to isolation of a strongly anticonvulsant isomeric mixture of E/Z isoxylitones (FIG. 1).

The screening of AED is based on mainly two types of tests i.e., acute seizure model (anticonvulsant activity) and chronic seizure model (anti-epileptogenesis activity). MEST and scPTZ tests are most commonly used acute seizure models whereas, the kindling model of epilepsy is considered to be a chronic model of epilepsy, which is primarily used for evaluating the test drug for anti-epileptogenic activity. The scPTZ test is widely used in AED discovery screening program. It evaluates the ability of the test substance to raise the seizure threshold for excitation of neural tissue. This test is most commonly used in the primary screening for new AEDs. Almost all the drugs active in scPTZ test demonstrate some clinical efficacy against myoclonic seizures which suggests that the PTZ test has a greater utility in the identification of drugs effective in myoclonic, rather than absence seizures. In the present study we used acute model of scPTZ-induced seizure and kindling model of epilepsy to evaluate the anticonvulsant and anti-epileptic activity of isoxylitones and its analogues.

The kindling is the model of epileptogenesis and was described by Graham Goddard in 1967. Kindling model became a focus of intense investigation due to two main reasons; firstly, this model provides several interesting properties that are of practical and theoretical significance, secondly, it's important to the clinical relevance along with neuroplastic phenomenon. Thus this model is now a very good screening tool for developing the new drug due to its universality across species and parameters throughout the brain. Usefulness of this model of human epilepsy is now well established.

### BRIEF SUMMARY OF THE INVENTION

Recently we have evaluated the effects of novel anticonvulsant isomeric compounds isoxylitones on the c-Fos protein and mRNA expression in the brain samples of kindled mice and compared it with the normal and untreated kindled groups. The isoxylitone (30 mg/kg)-treated group demonstrated significant reduction of c-Fos expression compared to PTZ-kindled control animals. Thus isoxylitones was found to have the capacity to control the seizures by mechanism such as the suppression of c-Fos protein and mRNA levels in different regions of the brain.

Among various neurotrophic factors, BDNF is the most potent factor required for neurogenesis and is necessary for peripheral and central nervous system development, maintenance and response to injury. The levels of these factors are tightly controlled in a tissue-specific manner. In addition to its normal physiological role, BDNF has also been suggested to be involved in various neurodegenerative pathologies including epileptogenesis. Levels of both BDNF mRNA and BDNF protein are known to be up-regulated in epilepsy. Since BDNF modulates excitatory and inhibitory synaptic transmission by inhibiting GABA_{A}-receptor mediated post-synaptic currents, it provides a potential mechanism for the observed upregulation. It has also been suggested to be involved in mossy fiber sprouting (MFS) pathway which is one of the underlining mechanism of epileptogenesis induced plasticity. BDNF not only promote the dendritic outgrowth of cortical neurons but also initiate long-term potentiation of excitatory synaptic transmission and thus these structural and synaptic plasticity have been implicated in epileptogenesis. Since BDNF also plays an important role in the neuronal plasticity associated with epilepsy therefore, our present study we have evaluated the effect of the test compounds on the expression levels of BDNF.

### BRIEF DESCRIPTION OF THE SEVERAL VIEWS OF THE DRAWINGS

FIG. 1 depicts analogues of Isoxylitones

FIG. 2 depicts mortality testing of isoxylitones following acute scPTZ-induced mortality test. Animals receiving isoxylitones 15 and 20 mg/kg exhibited 33.3% and 20.8% mortality whereas at the dose of 30 mg/kg it exhibited 0% (i.e., 100% protection) in the treated group.

FIG. 3 depicts effect of isoxylitones on scPTZ-induced kindling. The kindling scores of n=12/treatment group are expressed as arithmetic mean±SEM. The isoxylitones did not exhibit seizure score until the end of the kindling experiment. The timeline of the experiments is represented by the bar under the figure. The rats were given PTZ or saline treatment on alternate days. The animals in the control group were fully kindled after 18 doses were given during the 34-days period. The positive control kindled rats exhibited significantly higher average seizure scores starting from the 5th injection of PTZ onward. *p<0.05: significantly higher than normal control revealed by nonparametric Mann-Whitney U tests. The animals were sacrificed after 34 days and brain samples were collected for immunostaining.

FIGS. 4A and 4B depict effects of isoxylitones on BDNF protein expression following sc-PTZ-induced kindling. (A); the normal control group showed very little to no BDNF immunoreactivity. (B); PTZ kindling significantly increased the BDNF immunoreactivity in hippocampus and cortex regions (^{∞}<0.002 and ^{±}p<0.03, respectively). (C); the diazepam treatment reduced the levels to controls (^{δ}p<0.001, and ^{σ}p<0.02). (D); Isoxylitones treatment returned the enhanced BDNF immunoreactivity from PTZ kindling to the normal control levels in hippocampus (*p<0.03) and cortex (**P<0.01).

### DETAILED DESCRIPTION OF THE INVENTION

NMR spectral analyses were experimented on Avance Bruker AM 300-500 MHz Instrument. Mass spectrometric analyses EI-MS were done on a Finnigan MAT-311A, Germany. Thin layer chromatography (TLC) was carried out on pre-coated silica gel aluminum cards (Kieselgel 60, 254, E. Merck, Germany). Thin layer chromatograms were visualized by UV at 254 and 365 nm.

### Experimental Procedures

### Synthesis of E/Z Mixture of Conjugated Ester 2

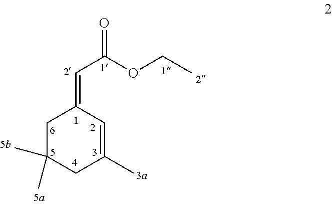

Triethyl phosphonoacetate (0.2 mmol, 34.5 mL) was added dropwise at 0° C. to the slurry of 50% NaH (0.2 mmol, 4.8 g) in dry THF (200 mL), the reaction mixture was stirred for 1 hour at room temperature until gas evolution had ceased. Isophorone (1) (0.1 mmol, 15 mL) was added drop wise. After addition, the mixture was refluxed for 2 days. The reaction mixture was taken in excess of water and the conjugated ester 2 was collected from organic layer, the product was purified on silica gel column eluted with a mixture of hexane:EtOAc (8:2). The product was identified by ESIMS and ¹HNMR spectral studies as E/Z mixture of ester 2.

¹HNMR, (CD₃OD, 300 MHz): Z-Isomer, δ 5.39 (1H, bs, H-2′), δ 4.073 (2H, q, H-1″), δ 1.22 (3H, t, H-2″), δ 7.29 (1H, bs, H-2), δ 1.85 (3H, bs, H-3a), δ 2.0 (2H, bs, H-4), 0.92 (6H, s, H-5a and H-5b), 2.10 (2H, d, J=0.9 Hz, H-6). E-Isomer, δ 5.55 (1H, bs, H-2′), δ 4.073 (2H, q, H-1″), δ 1.22 (3H, t, H-2″), δ 5.95 (1H, bs, H-2), δ 1.82 (3H, bs, H-3a), δ 1.97 (2H, bs, H-4), 0.93 (6H, s, H-5a and H-5b), 2.68 (2H, d, J=1.5 Hz, H-6). EI-MS m/z (relative abundance %) 208 (M+, 41.6), 193 (100), 163 (39.6), 147 (40.0), 119 (54.0).

### Synthesis of Isomeric Mixture of (E/Z) Acid Analogue 3

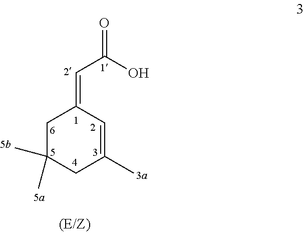

An E/Z mixture of ester 2 (28.8 mmol, 6 g) and 20.2 mL 40% aqueous KOH in 50% aqueous ethanol was stirred at 55-60° C. for 21 hours. At this stage, the pH of the reaction mixture was about 9 to 10. The reaction mixture was concentrated to remove excess of EtOH, and the resulting aqueous suspension was extracted with ethyl acetate. The ethyl acetate layer was dried over rotary evaporator and the aqueous layer was acidified with 10% solution of HCl. The acid was precipitated out and was isolated by filtration and dried. In the ethyl acetate layer the Z-isomer of the isomeric mixture of the acid 3a was crystallized out, while the aqueous layer contained (E/Z) isomeric mixture 3ab in a 2:1 ratio.

¹HNMR, (CD₃OD, 300 MHz) of 3: Z-Isomer, δ 5.38 (1H, bs, H-2′), δ 7.27 (1H, bs, H-2), δ 1.84 (3H, bs, H-3a), δ 2.0 (2H, bs, H-4), 0.93 (6H, s, H-5a and H-5b), 2.10 (2H, d, J=0.9 Hz, H-6). EI-MS m/z (realtive abundence %), 180 (M+, 60.6), 165 (100.0), 147 (26.1), 119 (72.2), 93 (35.1). E-Isomer, δ 5.54 (1H, bs, H-2′), δ 5.95 (1H, bs, H-2), δ 1.82 (3H, bs, H-3a), δ 1.97 (2H, bs, H-4), 0.93 (6H, s, H-5a and H-5b), 2.68 (2H, d, J=1.5 Hz, H-6). EI-MS m/z (relative abundance %), 180 (M+, 82.9), 165 (100.0), 147 (34.9), 119 (98.7), 93 (44.2).

### Synthesis of E/Z Mixture of Wienreb Amide (4)

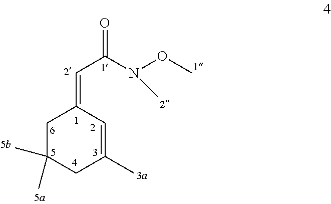

To the solution of acid 3 (0.25 mmol, 45 mg) in DCM, was added DMAP, (0.25 mmol, 30.5 mg), DIC, (0.375 mmol), N,O-dimethylamine hydrochloride (0.375 mmol, 36.5 mg), and Et₃N (0.375 mmol, 52.29 μL) and stirred overnight. The reaction mixture was then treated with 1M HCl, NaHCO₃, washed with brine and extracted with DCM. The organic layer was dried over MgSO₄. ¹HNMR, (CD₃OD, 300 MHz): Z-Isomer δ 5.91 (1H, bs, H-2′), δ 3.68 (3H, s, OCH₃), δ 3.19 (3H, s, —NCH₃), δ 7.13 (1H, bs, H-2), δ 1.82 (3H, bs, H-3a), δ 1.99 (2H, bs, δ 0.94 (6H, s, H-5a and H-5b), δ 2.13 (2H, d, J=0.9 Hz, H-6). E-Isomer, δ 5.98 (1H, bs, H-2), δ 3.68 (3H, s, OCH₃), δ 3.19 (3H, s, —NCH₃), δ □6.06 (1H, bs, H-2), δ□1.82 (3H, bs, H-3a), δ 1.97 (2H, bs, H-4), 0.92 (6H, s, H-5a and H-5b), 2.62 (2H, d, J=1.2 Hz, H-6). EI-MS m/z (relative abundance %) 223 (M+, 4.7), 164 (86.7), 163 (100.0), 135 (22.5), 107 (46.0), 93 (34.6).

### Preparation of E/Z Mixtures of Isoxylitones 5 and their Analogues from Weinreb Amide

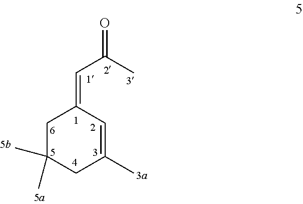

To the solution of amide 4 (1 g, 4.5 mmoles) in dry THF, the methyl magnesium bromide (45 mmol) was added at temperature below 0° C. The reaction mixture was first stirred for 30 minutes at lower temperature and then at room temperature for 60 minutes. An excess of saturated ammonium chloride solution was added to the reaction mixture and then was extracted with EtOAc and the organic layer was dried over anhydrous sodium sulfate. ¹HNMR, (CD₃OD, 300 MHz): Z-Isomer, δ 5.88 (1H, bs, H-1′), δ 2.15 (3H, bs, H-3′), δ 7.34 (1H, bs, H-2), δ 1.85 (3H, bs, H-3a), δ 2.02 (2H, bs, H-4), δ 0.93 (6H, s, H-5a and H-5b), δ 2.10 (2H, d, J=1.0 Hz, H-6): E-Isomer, δ 5.95 (1H, bs, H-1′), δ 2.17 (3H, bs, H-3′), δ 6.05 (1H, bs, H-2), δ 1.84 (3H, bs, H-3a), δ 1.99 (2H, bs, H-4), δ 0.92 (6H, s, H-5a and H-5b), δ 2.68 (2H, d, J=1.5 Hz, H-6): EI-MS m/z (relative abundence %) 178 (M+, 65.2), 163 (100.0), 145 (93.6), 105 (24.0), 91 (20.4).

The compounds 6-12 were prepared from Weinreb amide 4 and corresponding Grignard reagent through the procedure used in the synthesis of isoxylitones 5.

### 1′-(3,5,5-Trimethyl-2-cylohexen-1-ylidene)-2′-hexanone (6)

¹HNMR, (d₆-acetone, 300 MHz): Z-Isomer, δ 5.84 (H-1, bs, H-1′), δ 2.36 (2H, t, 3′-H), δ 1.52 (2H, q, H-4′), δ 1.307 (2H, q, H-5′) δ 0.87 (3H, t, H-6′) δ 7.41 (H-1, bs, δ 1.82 (3H, bs, H-3a), δ (2H, bs, H-4), δ 0.90 (6H, s, H-5a and H-5b), δ 2.07 (2H, bs, H-6)

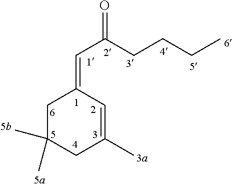 [SMILES: CCCCC(=O)/C=C1\C=C(C)CC(C)(C)C1]

E-isomer, δ 5.92 (1H, bs, H-1′), δ 2.36 (2H, t, 3′-H), δ 1.52 (2H, q, H-4′) δ 1.307 (2H, q, H-5′) δ 0.87 (3H, t, H-6′) δ 6.01 (1H, bs, H-2), δ 1.82 (3H, bs, H-3a), δ 1.98 (2H, bs, H-4), δ 0.90 (6H, s, H-5a and H-5b), δ 2.68 (2H, bs, H-6). EI-MS m/z (relative abundance %) 220 (M+, 14.2), 205 (22.42), 163 (100.0), 119 (23.7), 105 (21.9) 85 (36.6)

### 1′-(3,5,5-Trimethyl-2-cylohexen-1-ylidene)-2′-heptanone (7)

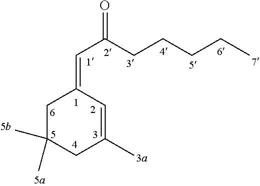 [SMILES: CCCCCC(=O)/C=C1\C=C(C)CC(C)(C)C1]

¹HNMR, (CD₃OD, 300 MHz) Z-Isomer, δ 5.87 (H-1, bs, H-1′), δ 2.42 (2H, m, H-3′), δ 1.57 (2H, q, H-4′), δ 1.29 (2H, m, H-5′), δ 1.29 (2H, m, H-6′), δ 0.89 (3H, m, H-7′), δ 7.33 (1H, bs, H-2), δ 1.85 (3H, bs, H-3a), δ 2.01 (2H, bs, H-4), δ 0.92 (6H, s, H-5a and H-5b), δ 2.09 (2H, d, J=0.9 Hz, H-6)

E-Isomer, δ 5.94 (1H, bs, H-1′), δ 2.42 (2H, m, H-3′), δ 1.57 (2H, q, H-4′), δ 1.29 (2H, m, H-5′), δ 1.29 (2H, m, H-6′), δ 0.89 (3H, m, H-7′), δ 6.04 (1H, bs, H-2), δ 1.85 (3H, bs, H-3a), δ 1.99 (2H, bs, H-4), δ 0.91 (6H, s, H-5a and H-5b), δ 2.67 (2H, d, J=1.5 Hz, H-6). EI-MS m/z (relative abundence %) 234 (M+, 14.0), 219 (24.3), 163 (100.0), 119 (14.9), 107 (8.7) 43 (59.5).

### 1′-(3″-Methylphenyl)-2′-(3,5,5-trimethyl-2-cylohexen-1-ylidene)-1′-ethanone (8)

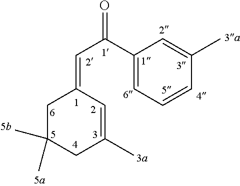

¹HNMR, (CD₃OD, 300 MHz): Z-Isomer, aromatic protons appeared in the upfield region between δ 7.33-7.71 as overlapped multiplets while the aromatic methyl group resonated at δ 2.39, δ 6.13 (1H, bs, H-2′), δ 7.40 (1H, bs, H-2), δ 1.87 (3H, bs, H-3a), δ (2H, bs, H-4), δ 0.97 (6H, s, H-5a and H-5b), δ 2.24 (2H, d, J=0.9 Hz, H-6): E-Isomer, aromatic protons δ 7.33-7.71 aromatic CH₃ δ 2.39, δ 6.56 (1H, bs, H-2′), δ 6.73 (1H, bs, H-2), δ 1.87 (3H, bs, H-3a), δ (2H, bs, H-4), δ 0.95 (6H, s, H-5a and H-5b), δ 2.76 (2H, d, J=1.5 Hz, H-6). EI-MS m/z (relative abundence %) 254 (M+, 30.2), 239 (30.5), 221 (100.0), 91 (36.6).

### 1′-(4″Methylphenyl)-2′-(3,5,5-trimethyl-2-cylohexen-1-ylidene)-1′-ethanone (9)

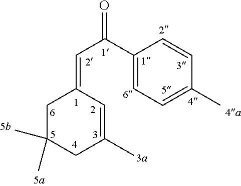

¹HNMR, (CD₃OD, 300 MHz): Z-Isomer, δ 7.83 (2H, d, J=8.1, H-2″ & H-6″), δ 7.28 (2H, d, J=7.8, H-3″ & H-5″), δ 2.38 (3H, s, H-4″a), δ 6.56 (1H, bs, H-2′), δ 7.38 (1H, bs, H-2), δ 1.87 (3H, bs, H-3a), δ (2H, bs, H-4), δ 0.97 (6H, s, H-5a & H-5b), δ 2.24 (2H, bs, H-6). E-Isomer, δ 7.83 (2H, d, J=8.1, H-2″ and H-6″), δ 7.28 (2H, d, J=7.8, H-3″ and H-5″), δ 2.42 (3H, s, H-4″a), δ 2.39, δ 6.13 (1H, bs, H-2′), δ 6.78 (1H, bs, H-2), δ 1.87 (3H, bs, H-3a), δ (2H, bs, H-4), δ 0.95 (6H, s, H-5a and H-5b), δ 2.75 (2H, bs, H-6). EI-MS m/z (relative abundance %) 254 (M+, 46.2), 239 (52.9), 221 (100.0), 119 (68.4), 91 (33.6).

### 1′-Cyclopentyl-2′-(3,5,5-trimethyl-2-cylohexen-1-ylidene)-1′-ethanone (10)

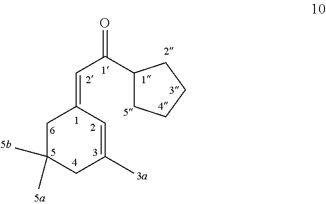 [SMILES: CC1=C/C(=C\C(=O)C2CCCC2)CC(C)(C)C1]

¹HNMR, (CD₃OD, 300 MHz): Z-Isomer, δ 2.99 (1H, q, H-1′), the methylene groups of the cylcopentyl ring were resonated in region between δ 1.59-1.84, δ 5.89 (1H, bs, H-2′), δ 7.33 (1H, bs, H-2), δ 1.84 (3H, bs, H-3a), δ (2H, bs, H-4), δ 0.93 (6H, s, H-5a & H-5b), δ 2.10 (2H, d, J=1.2 Hz, H-6). E-Isomer, δ 2.99 (1H, q, H-1′), CH₂ (C2″-C″) in range of δ 1.59-1.84, δ 5.95 (1H, bs, H-2′), δ 6.06 (1H, bs, H-2), δ 1.84 (3H, bs, H-3a), δ (2H, bs, H-4), δ 0.91 (6H, s, H-5a and H-5b), δ 2.66 (2H, d, J=1.5 Hz, H-6). EI-MS m/z (relative abundance %) 232 (M+, 52.5), 217 (26.2), 163 (100.0), 107 (26.6), 91 (24.1).

### 3′-Methyl-1′-(3,5,5-trimethyl-2-cylohexen-1-ylidene)-2′-butanone (11)

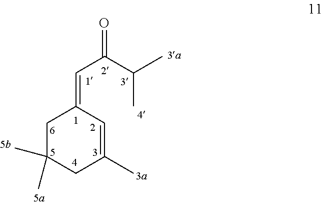

¹HNMR, (CD₃OD, 300 MHz): Z-Isomer, δ 5.91 (1H, bs, H-1′), δ 2.66 (1H, m, 3′-H), δ 1.07 (3H, d, J=6.9 H-3′a), δ 1.07 (3H, d, J=6.9 H-4′), δ 7.33 (1H, bs, H-2), δ 1.85 (3H, bs, H-3a), δ 2.01 (2H, bs, H-4), δ 0.93 (6H, s, H-5a and H-5b), δ 2.11 (2H, bs, H-6). E-isomer, δ 5.97 (1H, bs, H-1′), δ 2.66 (1H, m, H-3′), δ 1.07 (3H, d, J=6.9 H-3′a), δ 1.07 (3H, d, J=6.9 H-4), δ 6.08 (1H, bs, H-2), δ 1.85 (3H, bs, H-3a), δ 1.99 (2H, bs, H-4), δ 0.91 (6H, s, H-5a and H-5b), δ 2.66 (2H, bs, H-6). EI-MS m/z (relative abundance %) 206 (M+, 16.9), 163 (100.0), 107 (14.8), 83 (41.9).

### 4′-Methyl-1′-(3,5,5-trimethyl-2-cylohexen-1-ylidene)-2′-pentanone (12)

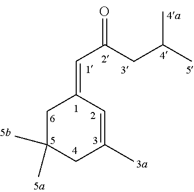 [SMILES: CC1=C/C(=C\C(=O)CC(C)C)CC(C)(C)C1]

¹HNMR, (CD₃OD, 300 MHz): Z-Isomer, δ 5.86 (1H, bs, H-1′), δ 2.28 (2H, m, H-3′), δ 2.09 (1H, m, H-4′), δ 0.92 (6H, m, H-4′a & H-5′), δ 7.34 (1H, bs, δ 1.85 (3H, bs, H-3a), δ 2.01 (2H, bs, H-4), δ 0.93 (6H, m, H-5a and H-5b), δ 2.09 (2H, d, J=1.2 Hz, H-6). E-Isomer, 5.94 (1H, bs, H-1′), δ 2.28 (2H, m, H-3′), δ 2.09 (1H, m, H-4′), δ 0.92 (6H, m, H-4′a and H-5′), δ 6.03 (1H, bs, H-2), δ 1.84 (3H, bs, H-3a), δ 1.99 (2H, bs, H-4), δ 0.92 (6H, m, H-5a and H-5b), δ 2.67 (2H, d, 1.5 Hz, H-6). EI-MS m/z (relative abundance %) 220 (M+, 18.0), 205 (22.2), 163 (100.0), 107 (16.8), 91 (22.0), 57 (49.2).

All compounds 1-12 were screened in in vivo models described in the following sections.

### Animals

Adult Male NMRI albino mice (strain acquired from Naval Medical Research Institute, Sweden) of weight 20-25 g were used for acute seizure model and chemical kindling model. The animals were housed at Animal House Facility, International Center for Chemical and Biological Sciences (ICCBS), University of Karachi. The mice were kept under environmentally controlled conditions having free access to standard laboratory food and water. The housing area temperature was maintained at 23±2° C. with the light/dark cycle of 12 hours each. To avoid circadian influence, all the experiments were performed between 9:00 am to 8:00 pm. The use of animals was approved by the Scientific Advisory Committee on Animal Care, Use and Standards, International Center for Chemical and Biological Sciences, University of Karachi, Pakistan, in compliance with the International Guidelines for the Care and Use of Laboratory Animals. Prior to the experimentations, the animals were acclimatized for 2-3 days with the experimental environment and with the experimenter. All the efforts were made to minimize stress to the animals and the group size was determined to the minimum number of animals for valid statistical analyses.

### Drugs/Reagents

The chemicals and reagents used for this study were of analytical research and standard laboratory grade. A chemical convulsant pentylenetetrazole (PTZ) was purchased from Sigma Chemical Company (St. Louis, Mo., USA) and the standard drug i.e., diazepam was a kind gift from Roche Pharmaceuticals (Roche Pakistan Ltd. Pakistan). All solutions were prepared freshly on the day of experiment.

Subcutaneous Pentylenetetrazole (scPTZ) Seizure Model (Acute Seizures Model)

Anticonvulsant effects of isoxylitones using scPTZ acute seizure test was evaluated by administering three doses i.e. 15, 20, and 30 mg/kg to groups of six mice, at least 30-40 min before subcutaneous administration of convulsive doses of PTZ (90 mg/kg). After administering PTZ, the mice were isolated and placed separately in a clear plexiglass chamber and closely observed for an hour for the presence or absence of different types of seizure patterns i.e. onset of body twitches, threshold seizures, generalized seizures with loss of righting reflex, loss of righting reflex with tonic forelimb seizures, loss of righting reflex with tonic forelimb and hind limb seizures (Table 1). The protection from PTZ-induced mortality was also monitored within 24 hours. In all experiments, diazepam (7.5 mg/kg i.p.) was used as a drug control.

TABLE 1
Behavioral rating scale for PTZ-induced epileptogenesis.

| Stages of Seizures | |
|---|---|
| (Score 1-5) | Seizure Pattern |
| 0 | No Response |
| 1 | Ear and facial twitching |
| 2 | Convulsive wave through the body |
| 3 | Myoclonic jerks |
| 4 | Clonic-tonic convulsions, turn over |
| | into side position |
| 5 | Generalized clonic-tonic seizures, |
| | turn over into back position |

Once screened in the acute seizure model, we next evaluated the effect of test compound on the development on epilepsy process using chemical kindling model of NMRI mice. Since we observed that the test compound at the dose of 30 mg/kg significantly retarded the acute seizures therefore, it was decided to use this dose for kindling experiments. The kindling was induced according to the modified method of De Sarro [23]. Briefly, animals were divided into four groups as shown in the Table 2. Each treatment group consists of 12 male NMRI mice ranging from 20-25 g. All the groups except normal control were given sub-convulsive dose of PTZ i.e., 50 mg/kg subcutaneously once on alternate day between 9:30-11:00 am. The normal control and drug control groups received daily intraperitoneal dose of saline (0.5 mL of 0.9% NaCl) and diazepam (7.5 mg/kg) respectively. Likewise, the test group was administered with the isoxylitones mixture (30 mg/kg, i.p.) once daily. On the day of PTZ administration, the treatment of saline, diazepam or isoxylitones were given 30-40 minutes before the PTZ. After each PTZ injection, animals were placed in observation chambers for 1 hour, and behavioral seizure activity was rated. Animals were scored according to a pre-validated scoring scale for the severity of the seizure activity they show. Seizure patterns during the gradual development of kindling are classified into five distinct behavioral stages as shown in the Table 1. The cumulative kindling score was then calculated and the experiments were terminated once the PTZ-control group reached the score 5. The brain samples were collected and processed for analyses of BDNF protein expression.

TABLE 2
Treatment groups of scPTZ-induced chemical kindling
model of epileptogenesis

| | | | Route of |
|---|---|---|---|
| Groups | Treatment | Dose | Administration |
| I (Normal Control) | Saline | 0.5 ml of 0.9% | i.p |
| II (Test group) | Isoxylitone + | 30 mg/kg | i.p. |
| | PTZ | | |
| III (Positive Control) | PTZ | 50 mg/kg | i.p |
| IV (Drug Control) | Diazepam + | 7.5 mg/kg | i.p. |
| | PTZ | | |

Brain samples from chemically kindled mice (as described above) were collected immediately after the last treatment of animals with PTZ. Mice were deeply anesthetized and transcardially perfused with ice-cold 50 mL solution containing phosphate buffer saline (PBS) and heparin [1 mL (5000 I.U.) of heparin added for 500 mL of PBS]. Brains were removed from cranial cavity and were gently rinsed in cold PBS. After washing, they were fixed in ice cold 4% paraformaldehyde solution for 24 hr at 4° C. These samples were then placed in a cryoprotectant i.e. 30% sucrose in PBS until they sank to the bottom of the container. The samples were subsequently stored at −80° C. until further processing. Cryostat sagittal sections were prepared using cryostat (Thermo Electron Corporation, UK), frozen brain sections of 30-μm thickness were cut at −20° C. and collected directly on poly-lysine coated slides. At the time of processing, the cryosections were kept overnight in a humid chamber at room temperature containing PBS buffer. Care was taken so that the slide having cryosections does not come in direct contact with PBS. On the following day, the sections were re-hydrated by rinsing three times (5 min/rinse) with PBS buffer, followed by incubation in blocking solution (pre-filtered with syringe filter of 0.45 μM size, consisting of 2% bovine serum albumin (BSA), 2% normal goat serum and 0.1% Tween-20) for 30 min at 37° C. For BDNF IHC, the sections were incubated overnight at 4° C. with the BDNF (N-20) primary antibody sc-546 rabbit IgG (Santa Cruz Biotechnology Inc., USA). Following overnight incubation in the primary antibody, sections were washed three times in PBS and then were incubated with secondary antibody, i.e. Alexa Fluor® 546 goat anti-rabbit IgG secondary antibody (1:100 dilution) from Invitrogen (Life Technologies, NY, USA) for 30 min at 37° C. in the dark, followed by a final washing step with PBS. Negative control slides were prepared without primary antibody to rule out the non-specific tissue binding of antibodies. The stained sections were observed under fluorescent microscope (Nikon ECLIPSE TE2000-S).

The schematic diagrams of brain sections adapted from the mouse brain atlas [24] were used as a visual guide for determining the sub-region boundaries. The image processing program ImageJ (National Institutes of Health, MD, USA) was used to analyze the images. This software helps in multiple imaging system data comparisons, taking density (densitometry) in consideration [25]. For each image, background density was determined and subtracted; the remaining particles were considered to represent BDNF expression. Data were obtained from two sections per rat (n=12 animals per group) and presented as means±S.E.M BDNF immunoreactivity in the amygdala, cortex, dorsal hippocampus and thalamus were centered approximately around 3.6 mm posterior to bregma. Within the hippocampus region, measurements were performed over the layer extending from sub-regions CA1-CA3.

The neurotoxic manifestation of isoxylitone was determined by inverted screen acute neurotoxicity test developed by Coughenour et al. in 1977. The apparatus consisted of six 13 cm square platforms of 0.6 cm wire mesh supported by metal bars mounted on a metal rod. The rod was supported at both ends and was inverted through an arc of 180°. Mice were pretested on the apparatus the day preceding the experiment, and those failing the task were not used for the subsequent drug test. Testing was carried out at 5, 30, 60 and 120 minutes following i.p. administration of 15 mg/kg, 20 mg/kg, 30 mg/kg, 50 mg/kg and 100 mg/kg of isoxylitone in groups of 6 mice. Mice unable to climb to an upright position for 1 min duration were rated as failures.

The behavioral analysis was performed adopting the modified procedure as described by Turner, 1972. The effects were recorded using a scoring system (scores were allocated according to the intensity of the symptoms from 0-4) as described by Turner i.e. for stereotype behavior 0: no effect; 1: intermittent; 2: continues 3: intense; 4: severe and for spontaneous activity 0: reduced activity; and 4: increased activity. Animals were transferred into individual cages to allow them to acclimatize to the new environment prior to the experiment. Animals were observed in these cages for 1-2 hr after isoxylitone treatment for the signs of following behavior:

| | |
|---|---|
| Hyper-locomotion | Head weaving |
| Biting/licking or grooming | Hyper-excitability |
| Ataxia | Sedation |

Blind-testing was employed i.e., the experimenter conducting this study was blinded to the treatment given to the animals in order to avoid any biased interpretations.

Muscle relaxant activity was examined by traction test. Forepaws of the mouse were placed on a small twisted wire rigidly supported above a bench top. Normal mice grasped the wire with forepaws and when allowed to hang free, placed at least one hind foot on wire within 5 seconds. The inability to put at least one hind foot on the wire was considered failure to the test. The test was conducted at 30 minutes and 1 hour after administration of diazepam and Isoxylitone.

The isoxylitone was tested for acute toxicity (LD₅₀) using Lorke's test. This method provides the acute toxicity data with the least consumption of animals i.e. initially only 12 animals are required; on obtaining the dose causing the death in 50% animals further three more animals is used at the same dose to get the data statistically significant. Briefly, animals were given different doses of isoxylitone i.e., 50 mg/kg, 75 mg/kg, 100 mg/kg, 500 mg/kg and 1000 mg/kg. After 24 and 48 hours, the maximum dose that had not induced mortality was considered as the maximum non-fatal dose. LD50 values and the corresponding confidence intervals were determined by the Litchfield and Wilcoxon methods (SPSS, version, USA). Data were expressed as mean values±SEM and tested with ANOVA and Tukey-Kramer tests.

After the administration of the compound in a group of six mice each, the animals were observed for gross behavioral effects. They were observed continuously for 2 hours after administration of the test compounds and then every 30 minutes for next 3 hours and finally after 24 hours. The CNS stimulation was judged by the following signs and symptoms:

| | |
|---|---|
| Locomotor Activity | Ataxia |
| Clonic & Tonic Convulsions | Sedation |
| Catalepsy | Crouching |
| Lacrymation | Salivation or signs which |
| | deviate from normal behavior |

The statistic was performed using Statistical Package for the Social Sciences (SPSS). Results are reported as Mean±SEM. Data is analyzed statistically using Student's t-test or one way ANOVA. Sequential differences among means were calculated at the level of P<0.05 using the SPSS version 10.

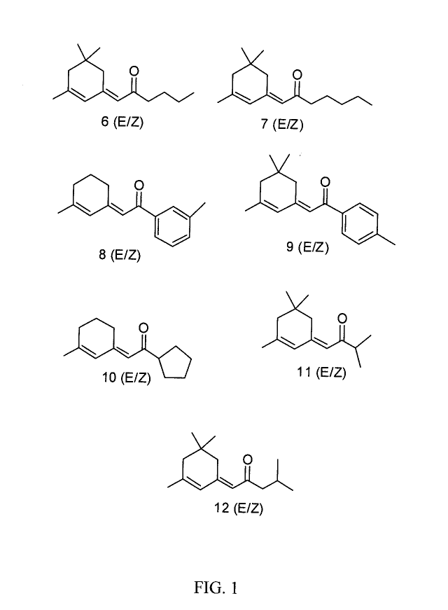

The total synthesis of anticonvulsant natural products isoxylitones and its structurally-related analogues was achieved through an improved synthetic strategy, their biological activity was evaluated. The synthesis of isoxylitones was started by using commercially available compound isophorone which was treated with phosphonate ester (Homer Wadsworth Emmons reaction) to obtain ester 2. which was simply hydrolyzed under basic conditions followed by simple amide synthesis which afforded the desired Weinreb amide 3. The compound 3 was treated with Grignard reagent (MeMgBr) which afforded the isomeric mixture of isoxylitones (E & Z) in 17% overall yield. This strategy has successfully eliminated the use of toxic chemicals such as Me3Al; originally reported by us in the total synthesis of isoxylitones [4]. The methyl group of isoxylitones was replaced by different aliphatic and aromatic substituents in analogues 6-12. The anticonvulsant activity of isoxylitones and its analogues was evaluated in vivo models. Among the compounds shown in FIG. 1, only isoxylitones and its acid analogue showed a strong anticonvulsant activity (scheme-1).

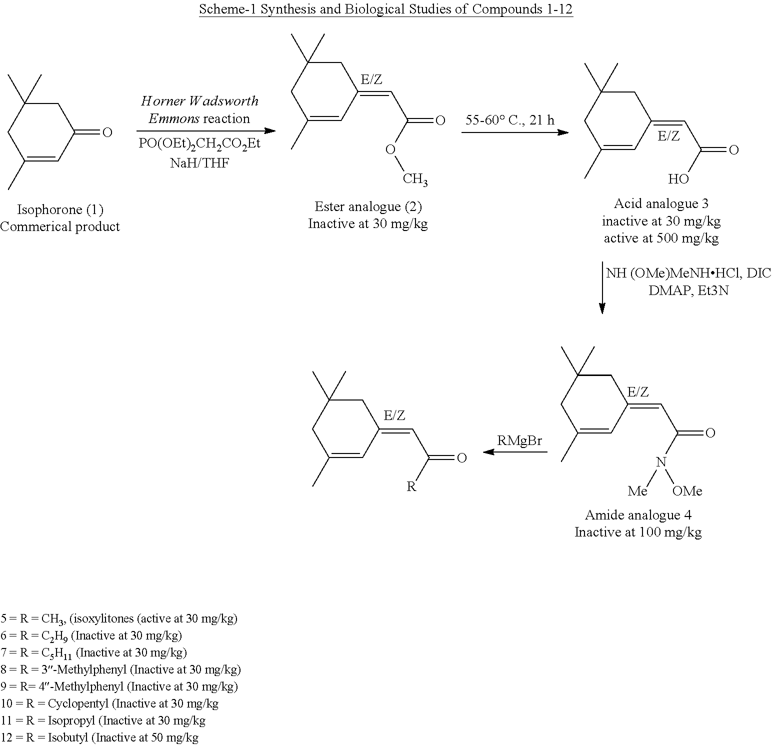

### In Vivo Anticonvulsant and Anti-Epileptogenenic Activity

Subcutaneous Pentylenetetrazole-Induced Seizure Test (scPTZ-Induced Acute Seizures Model):

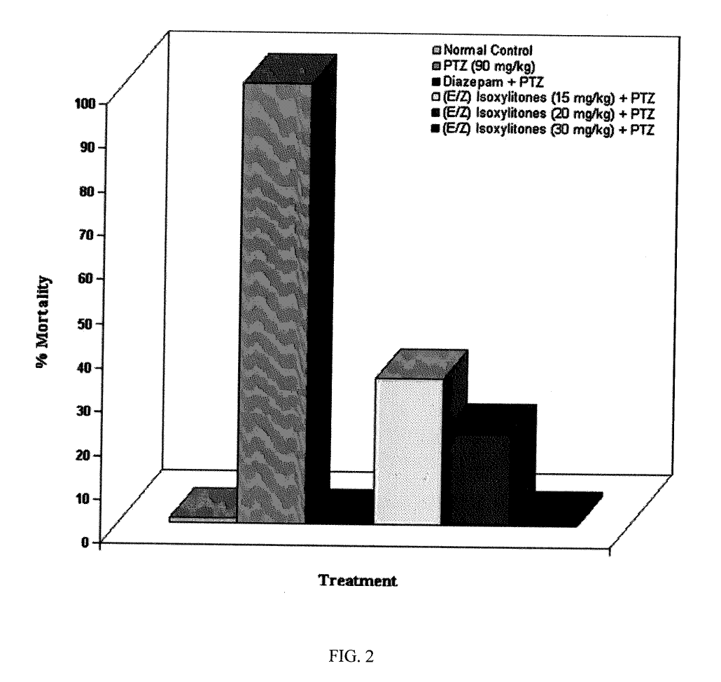

Isoxylitones 5 exhibited dose-related protection from different seizure patterns of PTZ-induced seizures i.e. twitches, body jerks, clonus and generalized seizure, in the animal group treated with isoxylitones prior to administration of PTZ. The E/Z isomeric mixture of isoxylitones was observed to effectively prevent PTZ-induced myoclonic twitches when tested at the dose of 15 and 20 mg/kg, and increasing the latency to first episode of seizures threshold; however, it was unable to provide complete protection from PTZ-induced seizure threshold (Table 3). Nevertheless, the dose of 30 mg/kg not only protected the myoclonic twitches but also provided complete (100%) protection from mortality and PTZ-induced loss of righting reflex with tonic-forelimb and tonic hind-limb seizures, which were comparable to that of the diazepam (7.5 mg/kg)-treated group. We also observed 100% mortality in scPTZ control group, whereas animals treated with 15 mg/kg and 20 mg/kg of isoxylitones exhibited 33.3% and 20.8% mortality, respectively (FIG. 2).

TABLE 3
Effect of isoxylitones on inhibition and duration of seizures and
mortality in acute scPTZ-induced seizures in mice. Both isoxylitones
(isomeric mixture of E/Z) and diazepam were injected i.p. 40-50 min
before the administration of 90 mg/kg of pentylenetetrazole
(s.c.). Values (n = 12) are presented as mean ± SEM for
the duration of tonic seizures.

| | | | Rearing | Hind Limb | Mortality |
|---|---|---|---|---|---|
| | | Onset of | & | Tonic | protec- |
| | | jerks | falling | Extension | tion |
| Group | Dose | (sec) | (sec) | (HLTE) (sec) | (%) |
| PTZ | 90 mg/kg | 200 ± 40 | 493 ± 75 | 875 ± 37 | 0 |
| Saline | 0.5 ml/kg | | | | 100 |
| (normal | | | | | |
| control) | | | | | |
| Isoxylitones | 15 mg/kg | 440 ± 55 | 990 ± 33 | 1260 ± 20 | 66.7 |
| Isoxylitones | 20 mg/kg | 580 ± 21 | 1120 ± 40 | 0 | 100 |
| Isoxylitones | 30 mg/kg | 900 ± 150 | 0 | 0 | 100 |
| Diazepam | 7.5 mg/kg | 850 ± 20 | 0 | 0 | 100 |

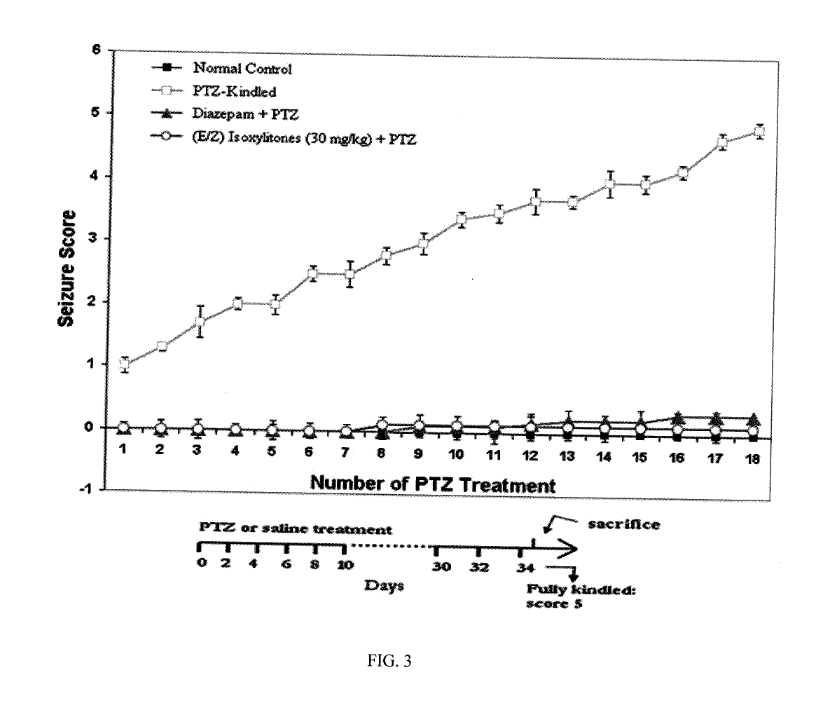

scPTZ-Induced Chemical Kindling Model of Epileptogenesis:
A gradual increase in the seizure score was displayed reaching a score of 5 after 18 treatments by the untreated scPTZ control group animals with an average seizure score of 4.9. The diazepam treated group compared to the PTZ-kindled control group did not exhibit any seizure pattern till the end of the kindling protocol. Based on the results of our previous experiments, it was decided to use only a 30 mg/kg dose of isoxylitones. At this test dose, isoxylitones exhibited a complete inhibition in the development of kindling induced by scPTZ administration (FIG. 3). Within the treatment groups, i.e., the isoxylitones (E/Z) and diazepam-treated animals, no difference was observed and both protected the animals from developing the seizures.

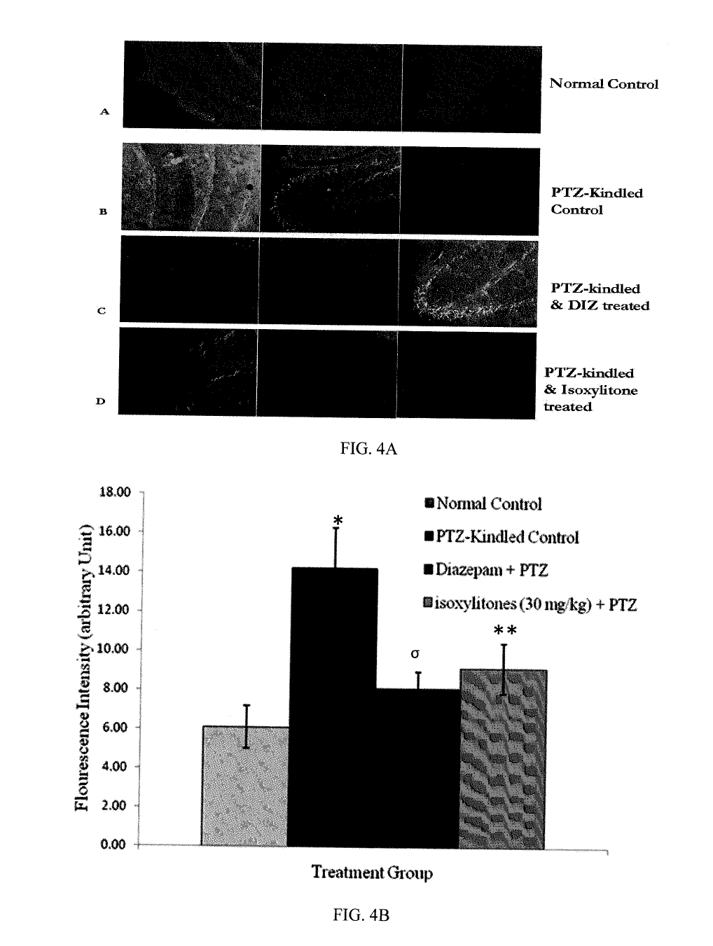

BDNF IHC was performed in the brains samples of controls and treatment groups as described in methodology section. The normal control group exhibited very little to no immunoreactivity in all the brain regions tested (FIG. 4). The PTZ administration significantly enhanced a homogeneous BDNF immunoreactivity in the polymorph inner layer (the stratum radiatum of the CA3/CA4 regions) of the hippocampus, and layers III, V and VI of the cortex region as compared to normal controls with ^{∞}p<0.002 and ^{±}p<0.03 respectively. In amygdala, kindled animals exhibited slight increase in the immunoreactivity; however it did not differ significantly from controls (p>0.05) and the levels remained comparable to controls in thalamus and hypothalamus. The pre-treatment with isoxylitones (30 mg/kg) returned the BDNF levels to normal levels in hippocampus and cortex with *p<0.03, and **P<0.01, respectively as compared to PTZ kindled group. The diazepam treatment also markedly reduced the immunoreactivity in these regions (^{δ}p<0.001, and ^{σ}p<0.02). The levels in the treatment groups (isoxylitones and diazepam) were observed comparable to the normal controls in all the regions examined.

Graphical representation of cumulative BDNF immunoreactivity analysed by ImageJ software in all four groups is shown at the bottom of the figure. PTZ kindling markedly increased BDNF protein expression as compared to controls p<0.02. The isoxylitones and diazepam treatments significantly suppressed PTZ-kindling induced upregulation of BDNF immunoreactivity with **P<0.03 and ^{σ}p<0.005, respectively.

None of the animals in the groups administered with 50 and 100 mg/kg of the isoxylitones showed signs of toxicity or altered behavior over a period of 24 hours. The animals receiving the dose of 250 mg/kg isoxylitones showed mild cramps and abdominal stretching approximately for 20 minutes after injection which then gradually subsides. No other CNS effects were observed. Likewise, the animals administered with 500 mg/kg dose of isoxylitones exhibited signs of discomfort and mild toxicity. Abdominal stretching was more pronounced at this dose. Ataxia, complete sedation and drowsiness were also manifested after 40 min of compound administration. Animals in this group returned to normal behavior over a period of 24 hrs. The animals receiving 1000 mg/kg exhibited complete ataxia and sedation. Animals were in a state of deep sleep for the next 4 hours and did not show any activity or reflexes. Mild recovery from sedation occurs after 4^{th} hr but ataxia was still present. Animal become normal after 24 hr. Due to these signs it was decided not to increase the dose further than 1000 mg/kg.

After the administration of the compound, the animals were observed for gross behavioral effects. They were observed continuously for 2 hours after administration of the test compounds and then every 30 minutes for next 3 hours and finally after 24 hours. The CNS stimulation was judged by the following signs and symptoms:

Locomotor activity, ataxia, clonic & tonic convulsions, sedation, catalepsy, crouching, lacrimation, salivation or any other signs which deviate from the normal behavior of the animal under observation.

Inverted screen acute neurotoxicity test was used in order to determine the effect of isoxylitones on motor function (Isoxylitones doses: 15, 20, 30, 50, 100 mg/kg, and time intervals: 5, 30, 60, 120 minutes) in mice. The compound did not show sign of acute neurotoxicity at any of the test doses and specified time.

There was no alteration in the spontaneous motor activity nor any of these observation were made i.e., ataxia, abdominal contractions, emesis, hyper-excitability, hyper-locomotion and twitches at the dose of 30 mg/kg, 50 mg/kg and 100 mg/kg.

The effective dose of isoxylitones i.e. 30 mg/kg was used in this study. The test was performed in SD rats. The animals divided into three groups and were daily administered with isoxylitones. The first group was sacrificed at the end of 15 days. The second group was sacrificed after 30 days and third group was sacrificed after 90 days. The rats were observed for morbidity and appearance of toxic signs and symptoms throughout the study period. At the end, blood was taken via cardiac puncture for biochemistry. Gross anatomical observations were also made for organs as liver, spleen, kidney, heart, and lung and spleen and to examine the organ abnormalities.

No mortality or morbidity was observed in any of the animals used throughout the 15-days, 30 days and 90 days observation period

There was no significant loss of fur and skin lesions. Nose and eyes appeared clear and normal. There was no diarrhea, convulsion, salivation, tremors, lethargy, sleep or coma which are signs associated with toxicity.

Animals did not show any sign of aggression or unusual behavior during handling

The serum levels total bilirubin, GPT, GOT, alkaline phosphatase, and LDH was estimated.

Isoxylitones showed no marked effect on the normal blood chemistry nor it has any detrimental effects on the normal functioning of the liver (measured in terms of sGPT and sGOT). The serum level of LDH was also within normal values which demonstrate that the isoxylitones did not cause any obvious damage to any cells/tissues.

The gross anatomical appearance of the kidneys, liver, heart, lungs and spleen was found to be normal in all three test groups.

1. A method of treatment for seizures comprising administering an effective amount of isoxylitone 2,2′-(3,5,5-Trimethyl-2-cylohexen-1-ylidene)acetic acid or an isomer, an acid analogue, a salt or a solvate thereof to an animal or human in need thereof.

2. The method of claim 1, wherein the seizures are associated with epilepsy.

3. A method of treatment for reducing the level of c-Fos expression comprising administering an effective amount of isoxylitone 2,2′-(3,5,5-Trimethyl-2-cylohexen-1-ylidene)acetic acid or an isomer, an acid analogue, a salt or a solvate thereof to an animal or human in need thereof.

4. A method of treatment for reducing the level BNDF protein expression comprising administering an effective amount of isoxylitone 2,2′-(3,5,5-Trimethyl-2-cylohexen-1-ylidene)acetic acid or an isomer, an acid analogue, a salt or a solvate thereof to an animal or human in need thereof.

5. The method of claim 1, wherein the isoxylitone further comprises a suitable physiological carrier.

6. The method of claim 3, wherein the isoxylitone further comprises a suitable physiological carrier.
